(19) United States
(12) Patent Application Publication
(10) Pub. No.: US 2026/0233220 A1
(43) Pub. Date: Aug. 13, 2026

(54) RUBBER INSERT FOR INSTRUMENT AND PIPETTE TIP SEALING AND ALIGNMENT

(71) Applicant: Coagulo Medical Technologies, Inc., Auburndale, MA (US)

(72) Inventors: Galit H. Frydman, Boston, MA (US); Gal Zeidman, Cambridge, MA (US); Rodney Scott Ziegenhagen, II, Bedford, NH (US); Alexander H. Slocum, Bow, NH (US)

(21) Appl. No.: 19/470,781

(22) PCT Filed: Mar. 26, 2024
(86) PCT No.: PCT/US2024/021452

§ 371 Date: Sep. 29, 2025

Related U.S. Application Data
(60) Provisional application No. 63/455,960, filed on Mar. 30, 2023.

(51) Int. Cl. B01L 3/00

(52) U.S. Cl. CPC B01L 3/502715; B01L 2200/027; B01L 2200/0689; B01L 2300/042; B01L 2300/123; B01L 2400/06

(57) ABSTRACT

Described herein are features and dimensions of an insert for an input port assembly. Also described herein are the advantages of such features and dimensions.

### FIELD OF THE INVENTION

The present invention generally relates to improved input ports for receiving pipette tips. Such input ports are useful when using a pipette to insert a fluid sample (e.g., a blood sample) into the inlet of a microfluidic device, for example.

### BACKGROUND OF THE INVENTION

Described herein are re-designed input ports that improve the way a fluid sample (e.g., a blood sample) is inserted into the inlet of a microfluidic cartridge. A conventional fixed diameter hard plastic port as illustrated in FIG. 1 requires users to insert a 200 μL pipette tip through a one-way slit valve and blindly locate a via hole, located at the bottom of the input port, that plugs into the microchannels of the cartridge. Without any proper tactile feedback or guide for the user to ascertain the location of this orifice, the users were found to dispense the fluid into the input port's dead space, where the fluid would accumulate and remain unused. In worst case scenarios, the fluid (in this case, a blood sample) that accumulated in the dead space would flow back out the one-way slit valve towards the user while the pipette tip was inserted, since the valve only holds pressure when fully closed. Only when the pipette tip was precisely oriented vertically could users dispense the fluid precisely into the orifice leading to the microchannels and reduce the hazardous risk of blood contacting the user.

The point-of-care microfluidic technologies currently available on the market do not offer a solution to the problem described above. Current technologies involve the following methodologies to insert a blood sample: using capillary action in an open system, as seen in paper-based microfluidics; molding a tapered conical plastic reservoir into the inlet port, meant to snugly fit the end of a standard pipette tip size and closing the input port after sample insertion to hold cartridge pressure; drilling a hole into a PDMS layer for insertion of a syringe or pipette tip. These methods do not work for inserting a sample into a closed microfluidic system and/or they can accommodate the insertion of only one pipette tip size. In addition, rubber inserts have been created for applications of alignment. One example is Sedi-rate, which uses a rubber plug tapered at the top to help align the insertion of one transfer pipette size in an open system. However, none of the devices currently available ensures proper alignment, tactile feedback, and sealing of multiple pipette tip sizes in a cartridge, while also eliminating dead space and user risks. The present invention addresses the need for such a device and provides a novel design of the input port assembly.

### BRIEF DESCRIPTION OF THE DRAWINGS

FIG. 1 shows a cross-sectional view of the previous design with the pipette tip fully inserted into the input port assembly for fluid ejection.

FIG. 2A shows a cross-sectional view of a cap, with modified ID taper to the cap.

FIG. 2B is a photograph of a 3D-printed cap with modified taper made out of Tough 2000.

FIG. 3A shows a cross-sectional view of an input port assembly, where the input port has an internal taper and the height of the input port is unchanged.

FIG. 3B shows cross-sectional view of an input port assembly, where the input port has an internal taper and where the height of the input port is increased (with a 4″ modification to the input port height) for improved tactile feedback.

FIG. 4A shows cross-sectional view of an input port assembly, with one O-ring below the valve.

FIG. 4B shows cross-sectional view of an input port assembly, with two O-rings below the valve and with a pipette tip inserted to demonstrate how tactile feedback can be improved.

FIGS. 5A-5D show cross-sectional view of updated O-ring design, showing how four different pipette tips fully engage into the input port, with a) Minivette 50 μL pipette tip, b) Minivette 20 μL pipette tip, c) Brandtech 200 μL pipette tip, and d) Oxford 200 μL pipette tip.

FIG. 6 shows a sketch of a foam washer concept with a cross-sectional view.

FIG. 7 shows a washer prototyped using the laser cutter and inserted into the input port.

FIG. 8 shows washer stack CAD model cross-section for prototyping concept.

FIG. 9A shows top view of CAD model of insert.

FIG. 9B shows cross-sectional view of CAD model of insert.

FIG. 10 shows CAD model of modified insert with tighter fit of pipette tip.

FIG. 11 updated design to make space for valve expansion as well as provide a taper directly after the valve for pipette tip insertion.

FIG. 12 shows a cross-sectional view of the three sections of the insert that were tested.

FIG. 13 shows a syringe-based pressurizing system for testing leakage.

FIG. 14 shows fixture for leak testing assembly with valve and rubber insert.

FIGS. 15A-15C show leak testing results and are graphs showing pressure changes versus time associated with number of insertions through the valve. FIG. 15A shows results associated with the 100 μL minivette pipette tip testing. FIG. 15B shows results associated with the 50 μL minivette pipette tip testing. FIG. 15C shows results associated with the 200 μL pipette tip testing.

FIG. 16 shows image of additional modifications to the insert.

FIG. 17 is an image of the fixture modifications, showing how the cap compresses the valve and insert assembly, reflecting more accurately how the assembly will operate in the cartridge. The NPT tapped hole is used to connect to the push-to-connect tube fitting that extends to the same test set-up described in FIG. 13.

FIGS. 18A-18B show leak testing results associated with two different cap and valve combinations used. FIG. 18A shows the results of the first cap tested. FIG. 18B shows the results of the second cap tested.

FIG. 19 is an image showing design change as described herein, with the input port step and mating insert step added to the assembly for improved support of the valve during pipette tip insertion.

FIGS. 20A-20C are rough drawings (in inches) of the critical dimensions associated with the insert (FIG. 20A), input port (FIG. 20B), and cap (FIG. 20C) within the assembly.

### DETAILED DESCRIPTION OF THE INVENTION

The drawings and embodiments of the invention described herein are provided by way of example, it being expressly understood that the description and drawings are only for the purpose of illustration and that the embodiments are not intended to define the limits of the present invention.

Described herein is a novel input port assembly that provides improved tactile feedback to the user, among other advantages. The novel input port as described herein can be used in a point-of-care medical diagnostics device, for example.

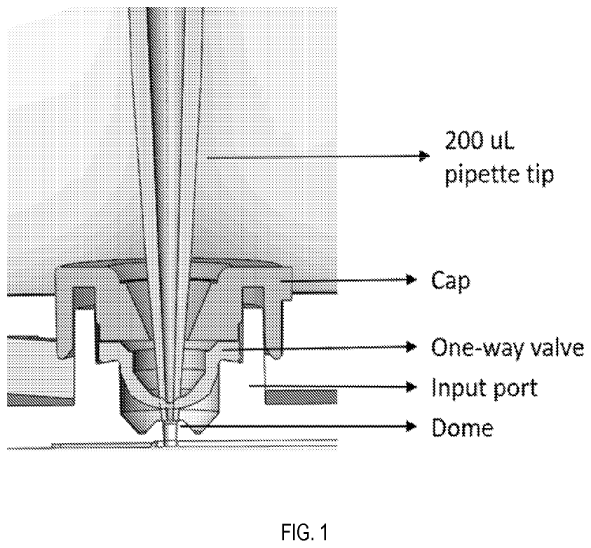

Previous design description: FIG. 1 shows a cross-sectional view of the previous design and the alignment of the pipette tip on top of the input port's dome that is required for the fluid within the pipette tip to be ejected into the orifice of the cartridge body. If any pipette tip misalignment occurs between the pipette tip and the dome due to the valve slit accommodating multiple insertion angles, the dispensed fluid could pile up in the dead space of the input port and flow back through the one-way valve while the pipette tip is inserted. The one-way valve is designed by MiniValve to hold 30 psi of pressure, and an injection molded cap is seated on top of the valve to hold it in place and ensure the valve's operation when the cartridge is pressurized.

A well-designed input assembly should satisfy the following requirements. The user should be able to insert multiple pipette tip sizes at a variety of insertion angles through a one-way slit valve designed to hold up to 30 psi of pressure, while being able to blindly align the end of the pipette tip precisely to form a seal to the via hole leading to the microchannels, without substantially increasing the force of insertion. The design should minimize dead space and regions where fluid could accumulate, which in turn will reduce the risk of fluid flowing back out the valve towards the user. Any fluid that does not go directly into the lower orifice should be prevented from reaching the top of the input port and contacting the user. Sufficient tactile feedback should be provided to ensure that the user dispenses the fluid sample at the appropriate height for reducing fluid ejection into dead space. In addition, the height of the input port should be minimized to ensure ease of packaging during large-scale production. Lastly, for tips that are inserted, a maximum diameter can be set to ensure the valve does not rupture (e.g., diameters smaller than 3.8 mm should be inserted through the valve).

### Design Process

Described herein are several strategies and prototypes that were subjected to blind user tests, with the aim of selecting a final design concept. Once the concept was selected, several additional blind user studies were conducted to finalize the design's internal and external dimensions. Leak testing was also performed to ensure that different sized pipette tips could be inserted into the assembly multiple times before the valve ruptured.

### Prototyping and Concepts

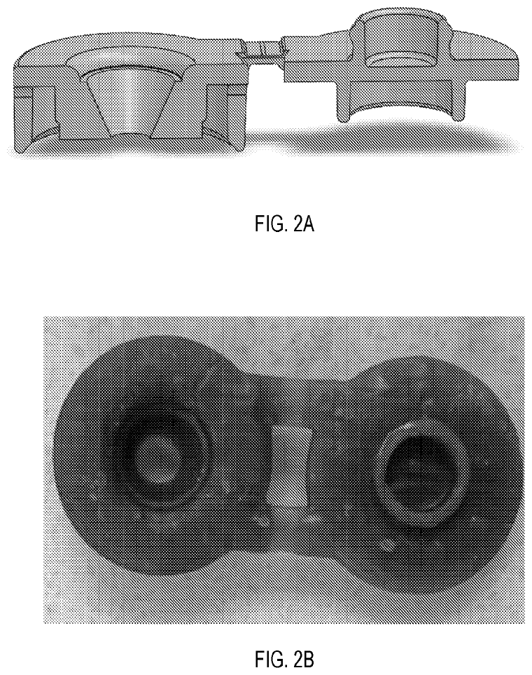

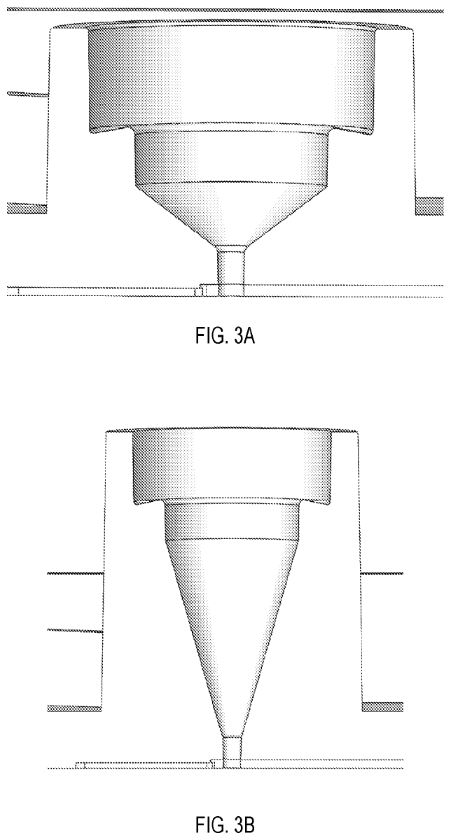

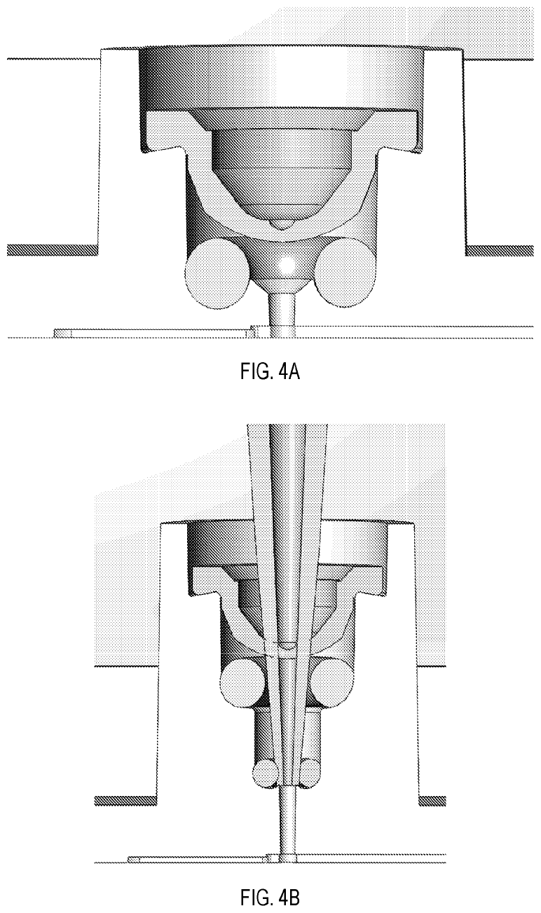

The following prototypes embodying different strategies for improving an input port assembly were made from Tough 2000 Resin 3D printed material: a taper molded into the bottom of the cap to guide the pipette tip before insertion into the valve and cartridge input port (FIG. 2); a taper molded into the cartridge input port for guiding the pipette tip after insertion of the tip into the valve (FIG. 3); one O-ring inserted below the valve for improved sealing (FIG. 4A); two O-rings inserted below the valve for improved tactile feedback and sealing (FIG. 4B).

Several observations were made during the testing of these prototypes. For example, having a funnel feature in the cap did not help in aligning the pipette tip to the bottom of the port. Users reported that they were unsure of the pipette location after the valve, and that they were still able to incorrectly position the pipette tip after the cap. In addition, for this design to accommodate multiple pipette tip sizes, a larger entry hole leading to the cap's taper would be required. As a result of a larger entry hole, smaller pipette tips would have a range of possible insertion angles that in turn would cause the dead space of the input port to fill up with liquid. Possible improvements to a design having a funnel in the cap (and before the valve) include using a smaller draft angle for the funnel while also providing a snug fit along the pipette tip; such a modification may require a taller input port.

The input port taper concept exhibited improved alignment of the pipette tip into the cartridge. The tapered input port prototype, with a larger total input port height, worked well. This prototype provided good sealing, was reported to be intuitive and easy to use. However, due to the absence of a sealing mechanism for constraining the pipette tip, the fluid could wick up the tip when the cartridge was pressurized and find its way out of the valve, especially when larger pipette tip sizes were used (larger pipette tips increase the dead space present). Users also reported that the increased height might increase the risk that the seal is broken; thus a possible improvement to this design is to reduce the extent to which the input port height is changed.

Finally, the O-ring concept showed great potential due to having both a compliant layer for sealing the pipette tip upon insertion, as well as ensuring a multitude of insertion angles could be used. In addition, the circular cross-section of the O-ring could provide improved tactile feedback by allowing users to slide into the correct orientation for contacting the via hole at the bottom of the input port. Due to these advantages, the O-ring concept was selected for further modification.

### Exploring the O-Ring Design Concept

Both O-ring strategies depicted in FIG. 4 were prototyped—the single O-ring design (FIG. 4A) and the two O-ring design (FIG. 4B). Having two O-rings improves tactile feedback by providing two points of contact along the pipette tip during insertion. However, the cost of manufacturing the two O-ring assembly is greater than the cost of manufacturing the single O-ring assembly. The single O-ring design was selected for further development, given the reduced cost of manufacturing this input port assembly compared to the cost of manufacturing the two O-ring design.

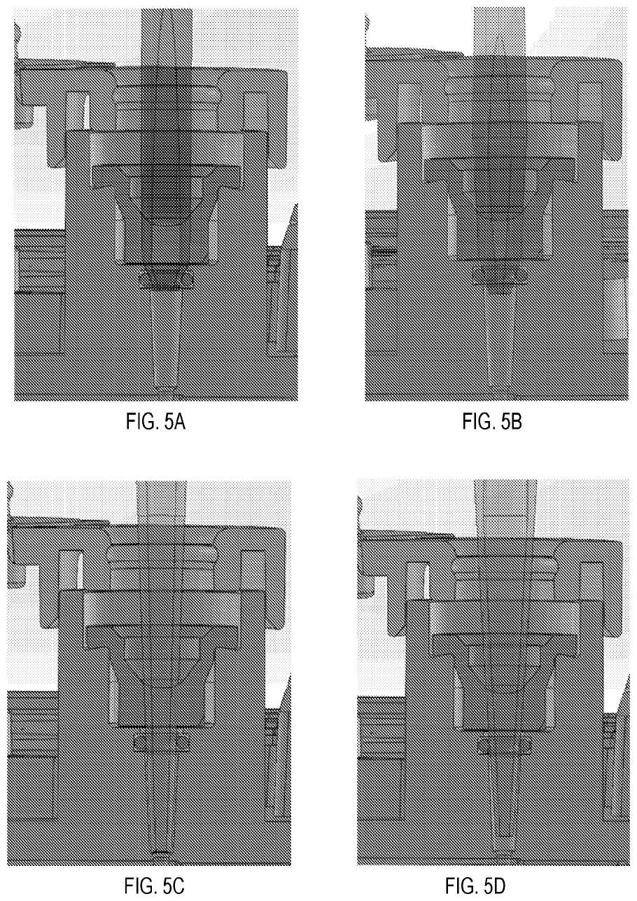

An input port was designed to fit a 0.065″ ID×0.022″ CS silicone 50A O-ring available from Apple Rubber (FIG. 5), and was designed to have the ability to receive four different pipette sizes, with each tip size resulting in a different amount of dead space.

The varying optimal depths of insertion can be seen in Table 1 to ensure an O-ring squeeze of less than 20% and a stretch larger than 5% during tip insertion. The chart also ensures that the diameter of the pipette tip through the valve at this optimal insertion depth was not larger than 3.8 mm.

TABLE 1
Optimal depth insertion using the selected Apple
Rubber O-ring, while ensuring that the diameter
of pipette at valve insertion is than 3.8 mm

| | Length from | Diameter CS of pipette | |
|---|---|---|---|
| | pipette tip end to | inserted at valve (must be | |
| Pipette brand | O-ring top plane | less than 3.8 mm) | |
| Brandtech 200 uL | 0.208″ | 1.85 | mm |
| Oxford 200 uL | 0.164″ | 2.23 | mm |
| Minivette 50 uL | 0.029″ | 2.57 | mm |
| Minivette 20 uL | 0.021″ | 2.6 | mm |

While this design allows multiple pipette tips to be inserted at different insertion angles, the pipette tip experiences contact only along the height of the O-ring. In addition, by minimizing the height of the input port, the larger Minivette tips will have a significant amount of dead space associated with insertion.

### The Washer Concept

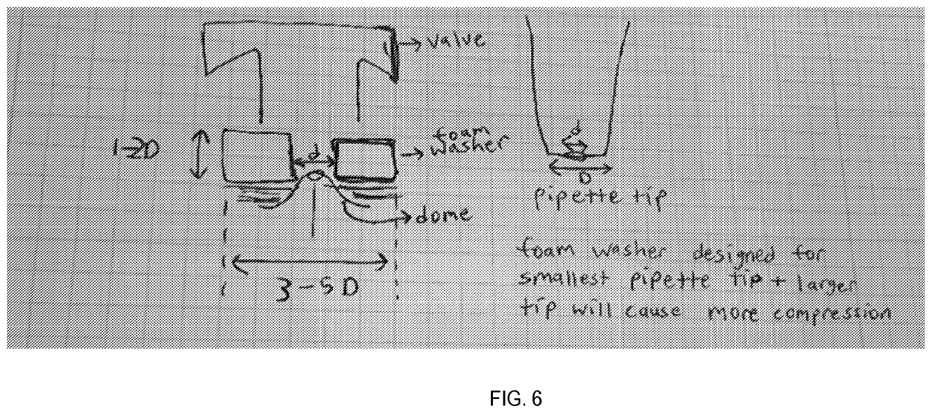

To compensate for the drawbacks observed with the O-ring concept, a thick foam washer concept was investigated next. It was hypothesized that the concept sketched in FIG. 6 could improve user experience by increasing the contact surface area, sealing pipette tips across multiple insertion diameters and angles, and minimizing input port height (FIG. 6).

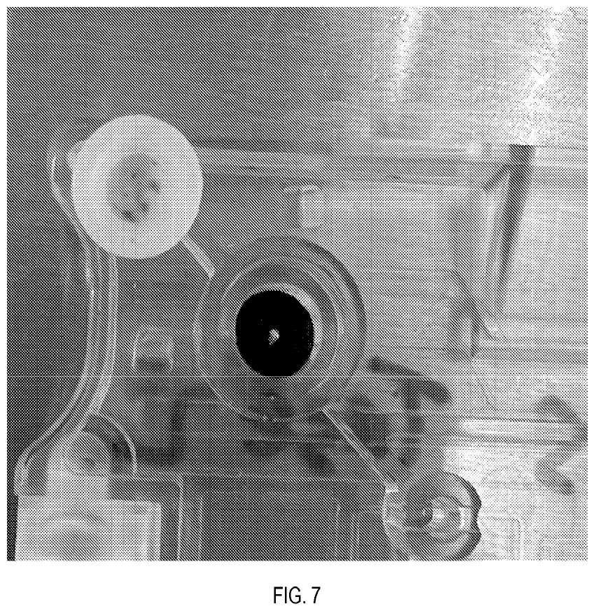

To prototype this concept, a 1 mm ID×5 mm OD foam washer was laser cut out of ultrasoft, ⅛″ thick neoprene material from McMaster, with the ID of the washer chosen to perfectly seal a small 200 μL pipette tip at standard input port height. Any larger pipette tips inserted were expected to radially compress the washer during insertion (FIG. 7).

As this prototype was tested in the lab, it was observed that users were sometimes unable to ascertain the location of the central cavity; due to the compliant nature of the foam, the users would sometimes assume that the top flat face of the washer was the central cavity, causing them to seal to the washer material itself rather than the inner diameter of the washer. Using a larger pipette tip did not improve these results because it was difficult for the user to find the ID of the washer and felt tactically better to seal to the flat of the surface. Sealing to the wrong surface, which was found to be easier to do at insertion angles offset from the vertical axis, would cause blood to pool up in the input port. These failed tests prompted an investigation to figure out what portion of the previous concepts could be incorporated into this design to improve alignment and guidance of the pipette tip into the input port. It was determined that providing a taper to the central cavity of the washer could provide both improved tactile feedback upon insertion through the usage of a soft compliant material as well as the ability for the user to be guided and aligned into the correct orientation and position.

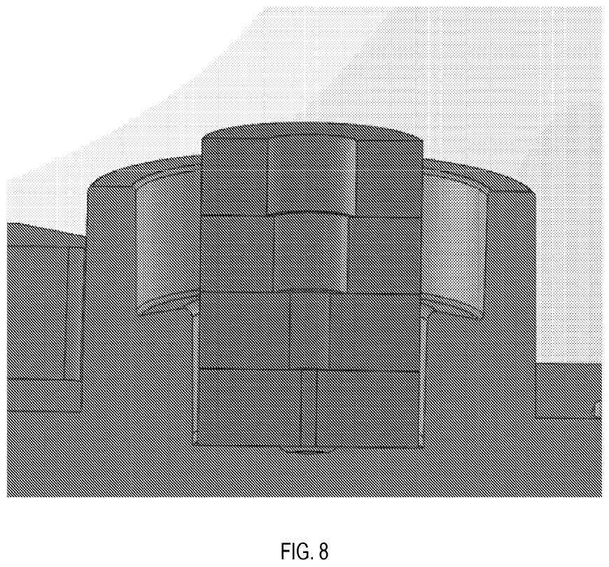

Accordingly, the next prototype included a 4-washer stack, 1/16″ in thickness each, to obtain a taper upon entry, with an entry ID 3× larger than the end of the pipette tip's diameter (FIG. 8). While improved results were observed with this prototype in terms of guiding and alignment of the pipette tip, prototyping with an incremental stack had its draw backs. Specifically, it was possible for the pipette tip at varying angles of insertion to seal into the flat of one of the washers in the stack, causing a hard stop that prevented liquid from being released through the pipette tip. This issue was resolved by creating a 3D printed part having a smooth taper across its internal cavity of a soft compliant material (instead of using an incremental stack with layered steps).

### Final Concept Selected: Rubber Insert Design

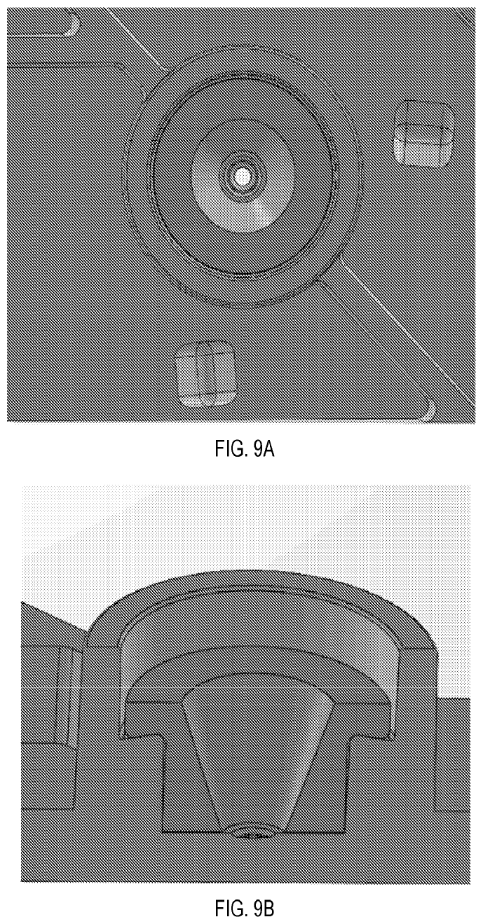

To prototype this design quickly with the cartridge currently available and without increasing the input port height, a new insert was developed to sit on top of the ridge usually reserved for valve placement (FIG. 9). The design was 3D printed out of TPU 70-A and tested in an open-system without the valve, to ensure that the tactile feedback associated with this concept was sufficient.

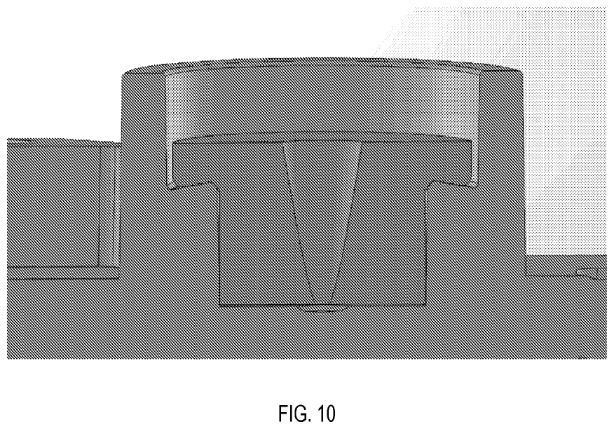

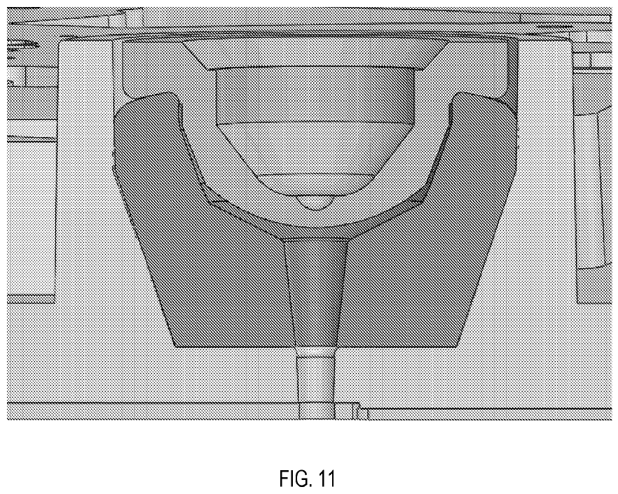

However, after testing this part, it was determined that the taper needed to have a tighter fit with the end of the pipette tip in order to increase tactile feedback, as shown in FIG. 10. The appropriate balance between (i) how large the entry hole could be for improving the guidance and alignment of the pipette tip upon its entrance into the insert, and (ii) ensuring the user felt a sufficient amount of friction and contact along the insert's height, needed to be obtained.

Lastly, placement of the valve in relation to the insert needed to be decided. A method was found both to make space for mating the one-way valve to the input port and to ensure the pipette tip would easily be able to find the central cavity, by removing the flat feature previously located at the top of the insert. FIG. 11 shows how the valve can be seated at the top of a rubber insert that is press-fit into the sides of the input port.

When the pipette tip is inserted into this new assembly, it will automatically be guided into the central cavity due to only being in contact with a taper along the sides. In comparison, the CAD model shown in FIG. 10 allowed for the possibility of the pipette tip to first contact the top flat surface rather than the internal taper during insertion. The tactile feedback associated with this design was deemed to be sufficient; the next step was to perform additional studies to finalize the internal and external dimensions of the insert.

### Selecting Dimensions of the Insert's Internal Cavity

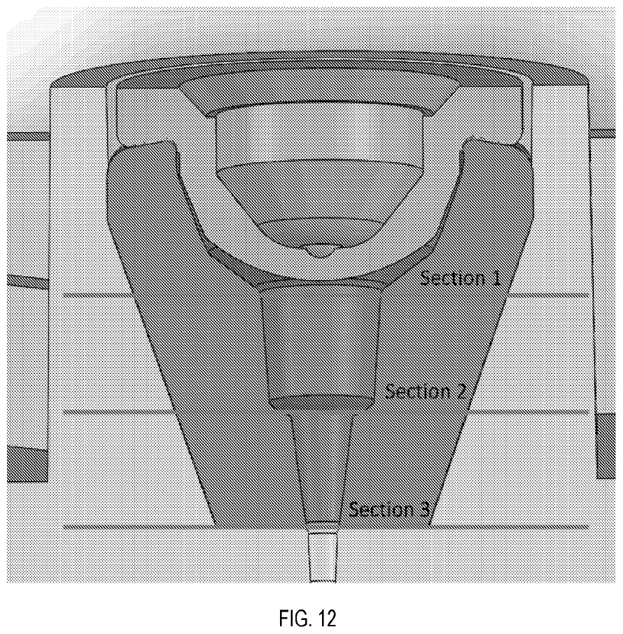

The following testing ensued to determine which dimensions of the insert's central cavity provided the best tactile feedback and performance. It was determined that there needed to be three sections of the insert (FIG. 12): section 1 would provide space for the one-way slit valve to open when a pipette tip is inserted, as well as an open guiding path for the pipette tip to find the central cavity; section 2 would be the region for insertion of the larger diameter minivette tips; section 3 would be the region for insertion of the smaller diameter 200 uL pipette tips as well as the dead space associated with dispensing fluid from the larger minivette tips. The central cavity associated with each of these three sections was tested independently, and the results were then combined into one insert, with the aim of minimizing the overall height of the input port.

### Blind User Testing Protocol for Selecting Features of Section Two and Section Three

A series of inserts with varying dimensions across the two sections were 3D printed and labeled. A user was subjected to blindly testing each of these inserts, selected at random from the batch to avoid biasing the user. Testing of each of these inserts consisted of receiving user feedback on how it felt to insert the pipette tip through the insert, valve, and cap assembly without simulated fluid. When the user feedback was positive regarding a specific insert, the two following tests were run: (1) dispensing a simulated fluid mixture into the assembly through an open cartridge system with a portion of microchannels in the 3D printed Accura 60 cartridge body; and (2) dispensing a simulated fluid mixture into the assembly through a closed cartridge system sealed with adhesive layers on both sides. These last two tests were performed to ensure that in both an open system and closed system, the pipette tip was able to dispense the fluid through the microchannels of the cartridge without the fluid flowing back through the one-way valve towards the user. These tests were run using the 100 uL minivette tip for section 2, and the 200 μL minivette tip for section 3. Performing tests with these two pipette tip sizes will enable usage of a range of other pipette tips that fall in between the tip ODs of the 100 μL minivette tip and the 200 μL minivette tip.

### Blind User Testing Results of 80A Insert

Table 2 shows the results of the study associated with section three of the insert, where taper angle and insert height were the parameters that were tested. These versions of the insert were 3D printed out of Flexible 80A material. The insert of the first row received the most positive user tactile feedback results as well as successful dispensing of fluid in closed and open cartridge systems. The inserts of the second and third rows exhibited tactile feedback that was satisfactory. The inserts of the remaining rows had poor tactile feedback results.

TABLE 2
User testing for improving the 200 uL pipette engagement with the insert
Testing of 200 uL pipette tip with 80A material

| | | Angle of taper | | |
|---|---|---|---|---|
| Length of insert taper | | 6.89 degrees | 6 degrees | 7.5 degrees |
| 1.861 | mm | V7 | | |
| 1.768 | mm | V7L.1 | | |
| 1.6749 | mm | V7L.2 | V7L.2A.1 | V7L.2A.2 |
| 1.5819 | mm | V7L.3 | | |
| 1.4888 | mm | V7L.4 | | |
| 1.3958 | mm | V7L.5 | | |
| 1.3027 | mm | V7L.6 | V7L.6A.1 | V7L.6A.2 |
| 1.20965 | mm | V7L.7 | | |
| 1.20965 | mm | V7L.8 | | |
| 1.1166 | mm | V7L.9 | | |
| 1.02355 | mm | V7L.10 | V7L.10A.1 | V7L.10A.2 |
| 0.9305 | mm | V7L.11 | | |
| 0.83745 | mm | V7L.12 | | |
| 0.7444 | mm | V7L.13 | | |
| 0.65135 | mm | V7L.14 | | |
| 0.5583 | mm | V7L.15 | | |
| 0.46525 | mm | V7L.16 | | |
| 0.3722 | mm | V7L.17 | | |
| 0.27915 | mm | V7L.18 | | |

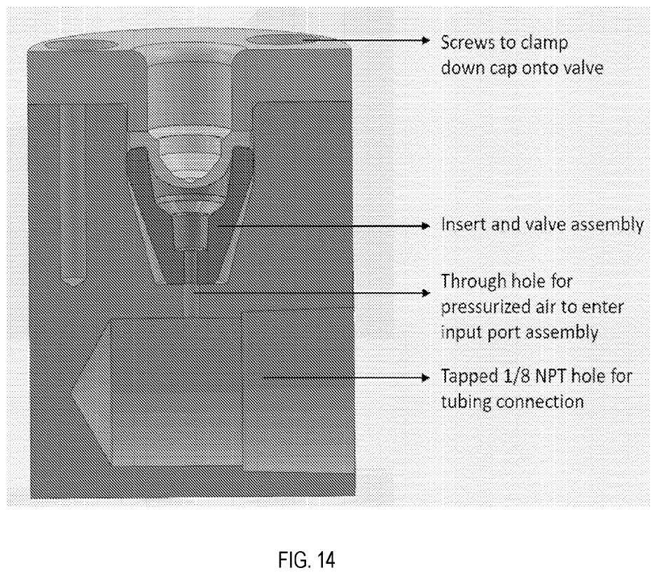

FIG. 14 shows a fixture for leak testing assembly with valve and rubber insert. For the studies performed on section two of the insert, two different methods were used to change the cavity's parameters. The first method involved changing the height of the tapered insert and taper angle (results provided in Table 3A), while the second method involved changing the entry ID, exit ID, and height of the taper (results provided in Table 3B). Of the inserts tested, NV1.10 in Table 3B exhibited excellent tactile feedback. NV1.8, NV.1.12, and NV1.13 (all in Table 3B) exhibited satisfactory tactile feedback. The tactile feedback of the remaining inserts described in Tables 3A and 3B were reported to be less than satisfactory, according to the user.

TABLE 3A
User testing for improving the 100 uL pipette engagement with the insert -
changing height of the tapered insert and changing taper angle
Testing of 100 uL minivette tip with 80A material

Angle of taper

| | | |
|---|---|---|
| Length of insert taper | 16.04 degrees | 15 degrees |

| | | | |
|---|---|---|---|
| 1.861 | mm | V1 | |
| 1.768 | mm | V1.1 | V1.1A1 |
| 1.6749 | mm | V1.2 | V1.2A1 |
| 1.5819 | mm | V1.3 | V1.3A1 |
| 1.4888 | mm | V1.4 | V1.4A1 |

TABLE 3B
User testing for improving the 100 uL pipette engagement with the
insert - changing the entry ID, exit ID, and height of the taper

| | Entry ID | Exit ID | Length |
|---|---|---|---|
| Part Name | (mm) | (mm) | (mm) |
| NV1 | 2.25 | 1.2 | 1.7 |
| NV1.1 | 2.27 | 1.4 | 1.7 |
| NV1.2 | 2.29 | 1.6 | 1.7 |
| NV1.3 | 2.23 | 1 | 1.7 |
| NV1.4 | 2.2 | 0.8 | 1.7 |
| NV1.8 | 2.265 | 1.8 | 1.861 |
| NV1.9 | 2.265 | 1.9 | 1.861 |
| NV1.10 | 2.265 | 2 | 1.861 |
| NV1.12 | 2.258 | 1.7 | 1.861 |
| NV1.13 | 2.258 | 1.6 | 1.861 |
| NV1.14 | 2.35 | 1.8 | 1.861 |
| NV1.15 | 2.35 | 1.9 | 1.861 |
| NV1.16 | 2.45 | 1.8 | 1.861 |

Combining the results of these two experiments and inserting those dimensions into one insert, and then testing it in a closed cartridge system with the valve fixed on top, showed promising results and sufficient user tactile feedback.

Blind User Testing Results with 50A Insert

The next experiment consisted of changing the hardness of the insert to 50A, with the goal of determining whether a more compliant material would provide better feedback. However, the dimensions finalized with the 80A material previously were not optimal for the 50A material prototyped. Accordingly, another round of experiments was performed to lock down the inner cavity dimensions with a more compliant material, which was hypothesized to result in improved user tactile feedback (Table 4). This time, instead of independently testing these two different sections of the insert, the dimensions of both sections were manipulated at the same time to increase the pace of 3D printing and testing of the designs using both pipette tips. Of these inserts tested, S10 and S31 performed best and exhibited excellent tactile feedback. Next best were S3, S4, S5, S9, S11, S13, S15, S21, S22, S23, S24, S25, S32, S33, and S34.

TABLE 4
Testing of 200 uL and 100 uL pipette tips with 50A material

| | | | |
|---|---|---|---|
| Part Number | Entry ID (mm) | Exit ID (mm) | Length (mm) |

Testing of 200 uL pipette with 50A material

| | | | |
|---|---|---|---|
| S1 | 1.23 | 0.7 | 1.861 |
| S2 | 1.2 | 0.6 | 1.861 |
| S3 | 1.2 | 0.7 | 1.861 |
| S4 | 1 | 0.3 | 1.861 |
| S5 | 0.8 | 0.6 | 1.861 |
| S6 | 0.8 | 0.5 | 1.861 |
| S7 | 0.8 | 0.5 | 2 |
| S9 | 0.9 | 0.8 | 2 |
| S10 | 0.8 | 0.7 | 1.861 |
| S11 | 0.8 | 0.8 | 1.861 |
| S12 | 0.7 | 0.8 | 1.861 |
| S13 | 0.7 | 0.7 | 1.861 |
| S14 | 0.75 | 0.7 | 1.861 |
| S15 | 0.7 | 0.4 | 1.861 |

Testing for 100 uL minivette tip with 50A material

| | | | |
|---|---|---|---|
| S21 | 2.4 | 2.3 | 1.861 |
| S22 | 2.45 | 2.35 | 1.861 |
| S23 | 2.5 | 2.4 | 1.861 |
| S24 | 2.4 | 2.35 | 1.861 |
| S25 | 2.4 | 2.4 | 1.861 |
| S26 | 2.4 | 2.25 | 1.861 |
| S27 | 2.4 | 2.2 | 1.861 |
| S28 | 2.45 | 2.3 | 1.861 |
| S29 | 2.45 | 2.25 | 1.861 |
| S30 | 2.45 | 2.2 | 1.861 |
| S31 | 2.45 | 2.4 | 1.861 |
| S32 | 2.4 | 2.5 | 1.861 |
| S33 | 2.5 | 2.35 | 1.861 |
| S34 | 2.5 | 2.3 | 1.861 |
| S35 | 2.5 | 2.25 | 1.861 |
| S36 | 2.5 | 2.2 | 1.861 |
| S37 | 2.5 | 2.5 | 1.861 |
| S38 | 2.6 | 2.4 | 1.861 |
| S39 | 2.6 | 2.35 | 1.861 |
| S40 | 2.6 | 2.3 | 1.861 |
| S41 | 2.6 | 2.5 | 1.861 |
| S42 | 2.7 | 2.4 | 1.861 |
| S43 | 2.7 | 2.35 | 1.861 |
| S44 | 2.7 | 2.3 | 1.861 |
| S45 | 2.7 | 2.5 | 1.861 |

Dead Space Associated with Multiple Pipette Tip Usage

After the final dimensions for section two and three of the 50A insert were finalized, the dead space was calculated in SolidWorks using the measurement tool. The smaller pipette tips, such as the 200 μL tip, inserted through section one will have no dead space. The larger pipette tips, such as the 100 μL minivette tip, inserted until section 2 will have a dead space of 1.65 μL.

### Leak Test Set-Up

The next section details the pressure testing that was pursued to ensure that the valve was not getting caught on the sides of the insert and tearing after placing the pipette tip through the assembly. These experiments were done to ensure that the valve could still hold pressure after each insertion. Considering that this cartridge is a one-time use product, no more than two pipette tip insertions are expected from a user. Thus, any leak testing done after four insertions, with a safety factor of two, is supplementary and optional data collected.

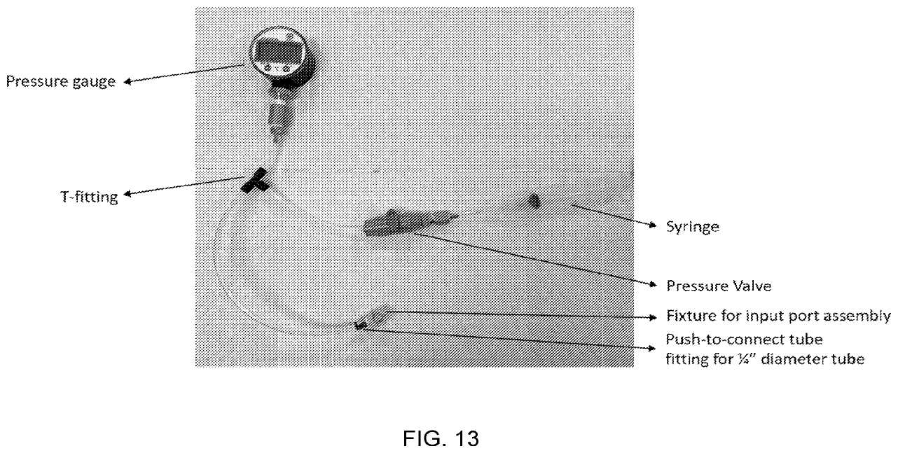

The leak testing fixture consists of pressurized air supplied by a syringe, which flows through a pressure valve and a series of tubes connected to both the fixture containing the input port assembly as well as a pressure gauge (FIG. 13) The fixture containing the input port assembly has an NPT tapped hole on its side that connects to a push-to-connect tube fitting for air to be pushed through the bottom of the assembly. FIG. 14 shows the cross-section of the fixture assembly, with the cap screwed down onto the valve from the top of the fixture to ensure that any leakage that occurs will happen through the valve.

Leak test procedure: All experiments were run by pressurizing the fixture for two minutes in water and taking note of the pressure value observed on the gauge every 15 seconds. If any excessive leakage occurred, bubbles could be seen emerging from the location where air was escaping out of the system. This procedure was tested on three different pipette tips, the 200 μL pipette tip, the minivette 100 μL pipette tip, and the minivette 50 μL pipette tip. The start of each experiment associated with one of these pipette tips included the following: Replacing the valve, running a control test to make sure leakage did not exist in other parts of the test set-up, and collecting a base line leak testing result before any pipette tips were inserted. The insertion angle of the pipette tip was not controlled for in this testing, which simulates how a user might not pay attention to the angle of insertion used.

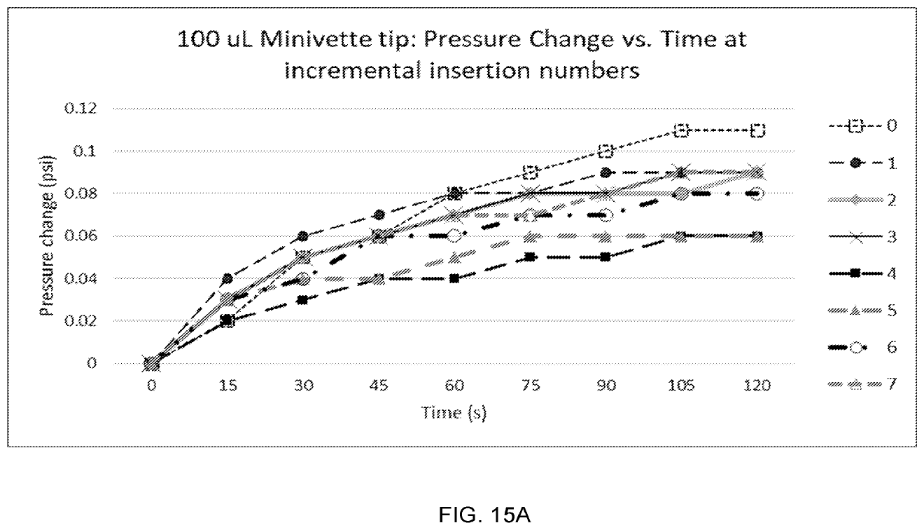

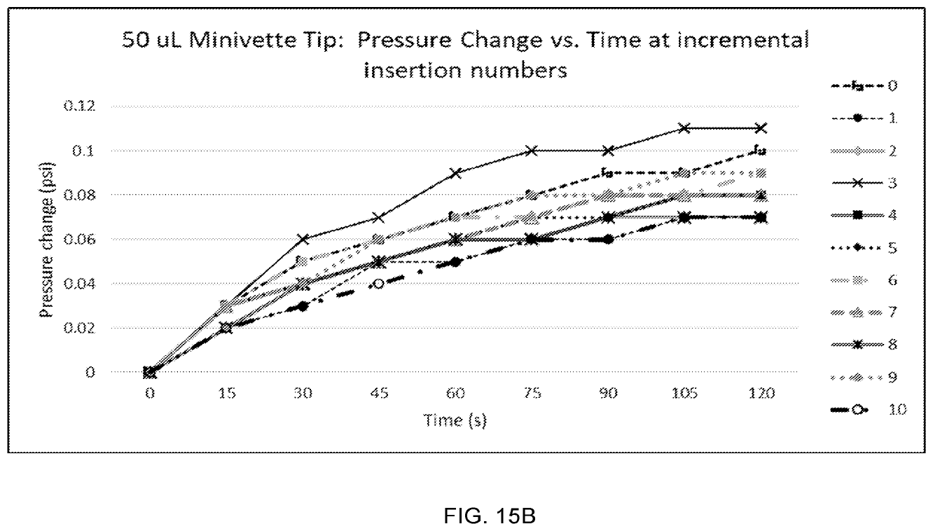

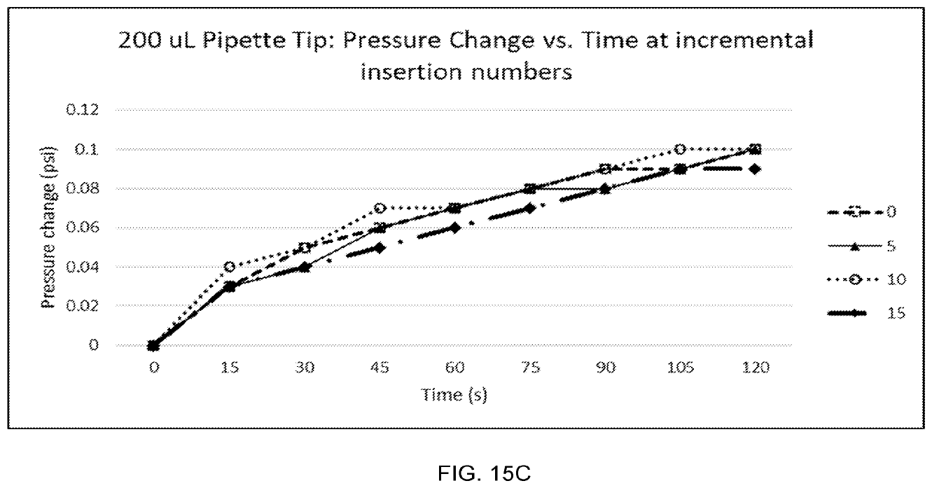

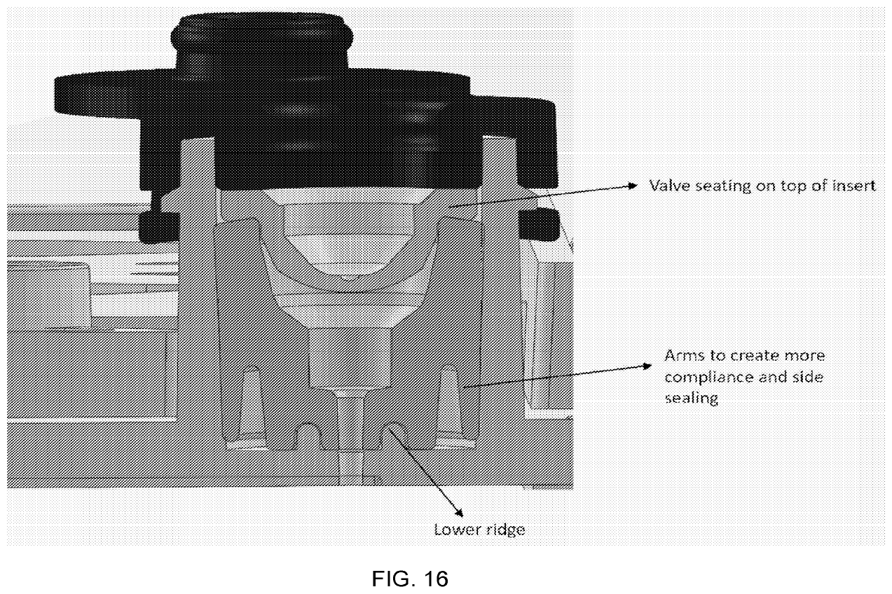

Leak test results: The raw data from testing is listed in Table 5. FIG. 15 shows the plotted results of the pressure change associated with a few of these insertions across the three different pipette tips, and in each figure, the pressure change does not surpass 0.12 PSI in 2 minutes. In the first minute, there is an expected initial pressure decay, but the graphs show increased stability past the 60 second mark. Specifically, less than a 0.1 PSI change/minute after this initial settling period is considered to be a successful leak testing result. Other results from this testing include the following: The 100 μL minivette survived 7 insertions before valve damage occurs; the 50 μL minivette survived 50 insertions without any valve damage; the 20 μL pipette tip could survive 20 insertions before valve damage occurs. The survival rates of the valve are different for these three pipette tips based off the cross-sectional diameter of the pipette tip through the valve at insertion. The larger the cross-sectional diameter of the pipette tip through the valve, the fewer times it can be inserted.

TABLE 5
Leak testing raw data

| | | |
|---|---|---|
| Number of insertions | Time (s) | Pressure (psi) |

100 uL minivette pipette tip valve testing

| | | |
|---|---|---|
| 0 | 0 | 15.54 |
| | 15 | 15.52 |
| | 30 | 15.49 |
| | 45 | 15.48 |
| | 60 | 15.46 |
| | 75 | 15.45 |
| | 90 | 15.44 |
| | 105 | 15.43 |
| | 120 | 15.43 |
| 1 | 0 | 9.9 |
| | 15 | 9.86 |
| | 30 | 9.84 |
| | 45 | 9.83 |
| | 60 | 9.82 |
| | 75 | 9.82 |
| | 90 | 9.81 |
| | 105 | 9.81 |
| | 120 | 9.81 |
| 2 | 0 | 11.21 |
| | 15 | 11.18 |
| | 30 | 11.16 |
| | 45 | 11.15 |
| | 60 | 11.14 |
| | 75 | 11.13 |
| | 90 | 11.13 |
| | 105 | 11.13 |
| | 120 | 11.12 |
| 3 | 0 | 14.37 |
| | 15 | 14.34 |
| | 30 | 14.32 |
| | 45 | 14.31 |
| | 60 | 14.3 |
| | 75 | 14.29 |
| | 90 | 14.29 |
| | 105 | 14.28 |
| | 120 | 14.28 |
| 4 | 0 | 10.29 |
| | 15 | 10.27 |
| | 30 | 10.26 |
| | 45 | 10.25 |
| | 60 | 10.25 |
| | 75 | 10.24 |
| | 90 | 10.24 |
| | 105 | 10.23 |
| | 120 | 10.23 |
| 5 | 0 | 8.57 |
| | 15 | 8.54 |
| | 30 | 8.53 |
| | 45 | 8.53 |
| | 60 | 8.52 |
| | 75 | 8.51 |
| | 90 | 8.51 |
| | 105 | 8.51 |
| | 120 | 8.51 |
| 6 | 0 | 11.69 |
| | 15 | 11.66 |
| | 30 | 11.65 |
| | 45 | 11.63 |
| | 60 | 11.63 |
| | 75 | 11.62 |
| | 90 | 11.62 |
| | 105 | 11.61 |
| | 120 | 11.61 |
| 7 | 0 | 13.79 |
| | 15 | 13.76 |
| | 30 | 13.74 |
| | 45 | 13.73 |
| | 60 | 13.72 |
| | 75 | 13.72 |
| | 90 | 13.71 |
| | 105 | 13.7 |
| | 120 | 13.7 |

50 uL minivette pipette tip valve testing

| | | |
|---|---|---|
| 0 | 0 | 15.98 |
| | 15 | 15.95 |
| | 30 | 15.93 |
| | 45 | 15.92 |
| | 60 | 15.91 |
| | 75 | 15.9 |
| | 90 | 15.89 |
| | 105 | 15.89 |
| | 120 | 15.88 |
| 1 | 0 | 11.99 |
| | 15 | 11.97 |
| | 30 | 11.96 |
| | 45 | 11.94 |
| | 60 | 11.94 |
| | 75 | 11.93 |
| | 90 | 11.93 |
| | 105 | 11.92 |
| | 120 | 11.92 |
| 2 | 0 | 13.35 |
| | 15 | 13.32 |
| | 30 | 13.31 |
| | 45 | 13.3 |
| | 60 | 13.29 |
| | 75 | 13.29 |
| | 90 | 13.28 |
| | 105 | 13.27 |
| | 120 | 13.27 |
| 3 | 0 | 8.37 |
| | 15 | 8.34 |
| | 30 | 8.31 |
| | 45 | 8.3 |
| | 60 | 8.28 |
| | 75 | 8.27 |
| | 90 | 8.27 |
| | 105 | 8.26 |
| | 120 | 8.26 |
| 4 | 0 | 10.24 |
| | 15 | 10.22 |
| | 30 | 10.2 |
| | 45 | 10.19 |
| | 60 | 10.18 |
| | 75 | 10.18 |
| | 90 | 10.17 |
| | 105 | 10.16 |
| | 120 | 10.16 |
| 5 | 0 | 10.54 |
| | 15 | 10.51 |
| | 30 | 10.5 |
| | 45 | 10.49 |
| | 60 | 10.48 |
| | 75 | 10.47 |
| | 90 | 10.47 |
| | 105 | 10.46 |
| | 120 | 10.46 |
| 6 | 0 | 8.92 |
| | 15 | 8.89 |
| | 30 | 8.87 |
| | 45 | 8.86 |
| | 60 | 8.85 |
| | 75 | 8.85 |
| | 90 | 8.84 |
| | 105 | 8.84 |
| | 120 | 8.83 |
| 7 | 0 | 12.18 |
| | 15 | 12.15 |
| | 30 | 12.14 |
| | 45 | 12.13 |
| | 60 | 12.12 |
| | 75 | 12.11 |
| | 90 | 12.1 |
| | 105 | 12.1 |
| | 120 | 12.1 |
| 8 | 0 | 12.61 |
| | 15 | 12.59 |
| | 30 | 12.57 |
| | 45 | 12.56 |
| | 60 | 12.55 |
| | 75 | 12.55 |
| | 90 | 12.54 |
| | 105 | 12.54 |
| | 120 | 12.54 |
| 9 | 0 | 13.98 |
| | 15 | 13.96 |
| | 30 | 13.94 |
| | 45 | 13.92 |
| | 60 | 13.91 |
| | 75 | 13.9 |
| | 90 | 13.9 |
| | 105 | 13.89 |
| | 120 | 13.89 |
| 10 | 0 | 12.8 |
| | 15 | 12.78 |
| | 30 | 12.77 |
| | 45 | 12.76 |
| | 60 | 12.75 |
| | 75 | 12.74 |
| | 90 | 12.74 |
| | 105 | 12.73 |
| | 120 | 12.73 |
| 15 | 0 | 12.74 |
| | 15 | 12.71 |
| | 30 | 12.7 |
| | 45 | 12.69 |
| | 60 | 12.68 |
| | 75 | 12.67 |
| | 90 | 12.67 |
| | 105 | 12.66 |
| | 120 | 12.66 |
| 20 | 0 | 14.01 |
| | 15 | 13.99 |
| | 30 | 13.97 |
| | 45 | 13.96 |
| | 60 | 13.95 |
| | 75 | 13.95 |
| | 90 | 13.94 |
| | 105 | 13.94 |
| | 120 | 13.93 |
| 25 | 0 | 14.38 |
| | 15 | 14.35 |
| | 30 | 14.33 |
| | 45 | 14.32 |
| | 60 | 14.3 |
| | 75 | 14.29 |
| | 90 | 14.29 |
| | 105 | 14.28 |
| | 120 | 14.28 |
| 35 | 0 | 10.6 |
| | 15 | 10.58 |
| | 30 | 10.56 |
| | 45 | 10.56 |
| | 60 | 10.55 |
| | 75 | 10.54 |
| | 90 | 10.54 |
| | 105 | 10.54 |
| | 120 | 10.54 |
| 50 | 0 | 13.44 |
| | 15 | 13.42 |
| | 30 | 13.41 |
| | 45 | 13.4 |
| | 60 | 13.39 |
| | 75 | 13.39 |
| | 90 | 13.38 |
| | 105 | 13.38 |
| | 120 | 13.38 |

200 uL pipette tip testing

| | | |
|---|---|---|
| 0 | 0 | 15.25 |
| | 15 | 15.22 |
| | 30 | 15.2 |
| | 45 | 15.19 |
| | 60 | 15.18 |
| | 75 | 15.17 |
| | 90 | 1.16 |
| | 105 | 15.16 |
| | 120 | 15.15 |
| 5 | 0 | 12.8 |
| | 15 | 12.77 |
| | 30 | 12.76 |
| | 45 | 12.74 |
| | 60 | 12.73 |
| | 75 | 12.72 |
| | 90 | 12.72 |
| | 105 | 12.71 |
| | 120 | 12.7 |
| 10 | 0 | 12.76 |
| | 15 | 12.72 |
| | 30 | 12.71 |
| | 45 | 12.69 |
| | 60 | 12.69 |
| | 75 | 12.68 |
| | 90 | 12.67 |
| | 105 | 12.66 |
| | 120 | 12.66 |
| 15 | 0 | 11.56 |
| | 15 | 11.53 |
| | 30 | 11.52 |
| | 45 | 11.51 |
| | 60 | 11.5 |
| | 75 | 11.49 |
| | 90 | 11.48 |
| | 105 | 11.47 |
| | 120 | 11.47 |

### Refinement of the External Features of the Insert

After performing tests relating to the insert's central cavity dimensions, focus was given to refining the external region of the insert. Specifically, press-fitting the insert along its height could over constrain the assembly or prevent the full seating of the insert into the base of the input port, without the ability for a technician or assembler to recognize it was occurring. Thus, it was decided to press-fit only the top third of the insert by 1% to the cavity of the input port and allow the bottom half to mate with a lower ridge through a 0.005″ clearance fit that would help to align and orient the part when the insert is pushed in (FIG. 16). This lower ridge also ensures that the orifice leading to the cartridge microchannels will always be aligned with the cavity of the insert during the assembly process as well as when a pipette tip gets inserted at varying angles. Lastly, in the event of blood leaking out past the cartridge via hole and through the sides of the insert, this lower ridge provides a lip to contain the fluid and makes it more difficult for the fluid to continue leaking up through the sides and past the insert or valve.

After re-designing the input port cavity to accommodate these changes to the insert, an additional feature to the insert was added to its external diameter to increase compliance during pipette insertion. This feature ensures that as larger diameter pipette sizes are used, there won't be a drastic change in the tactile feedback for a user or a large force required at insertion. Shown in FIG. 16 is an unconstrained section cut-out in the middle of the part where the initial insertions will first occur, allowing for the increased movement of pipette tips through the cavity, aided by the soft 50A flexible material. The side arms also compress by 3 degrees onto the input port to provide a second storage location for blood to pool up if it makes its way to the sides of the insert. This will prevent any leakage from reaching the cap on the sides, and the valve prevents any liquid from entering through the central cavity of the input port.

Leak Testing with Refined Insert and Cap

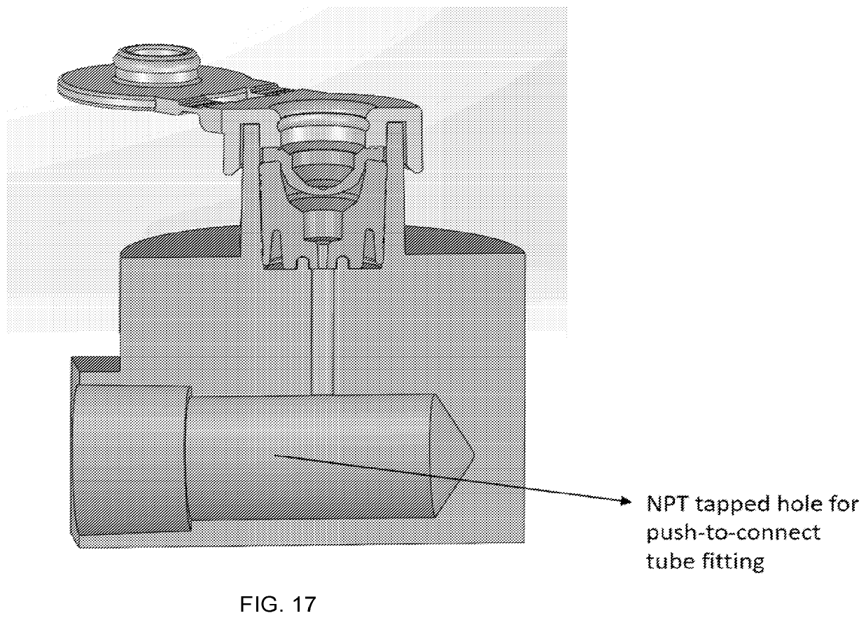

Using a similar procedure and test set-up as described above, leak testing was performed on the refined insert design, input port, and cap. The main change to the set-up included a modification to the fixture to allow testing of the cap in parallel. FIG. 17 shows how the fixture was modified to test the operation of the full assembly during leak testing.

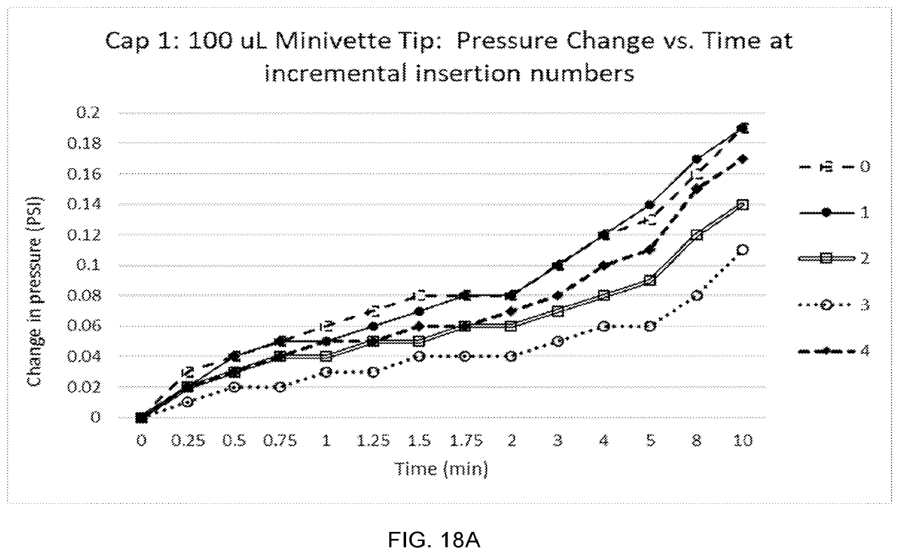

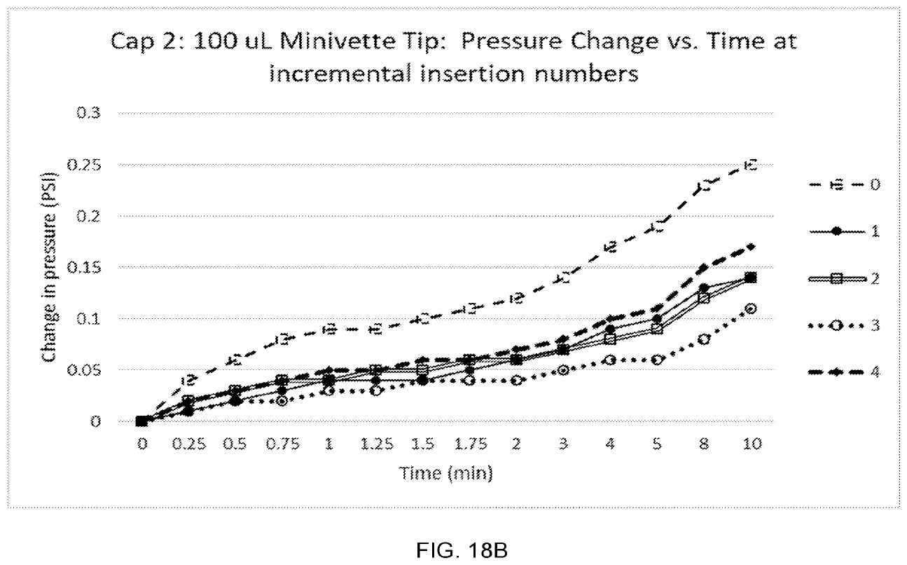

The results of these experiments are plotted in FIG. 18, with the raw data listed out in Table 6. Two separate caps and valves were used to perform leakage testing with the 100 μL minivette pipette tip, which provides worst-case leakage results due to this pipette tip having the largest cross-sectional diameter upon entering the valve in comparison to the other three tips available in-house. For each experiment, the data was collected after each insertion, with a total of four insertions conducted, contributing to a safety factor of 3 since the user is expected to insert a pipette tip into the assembly only once.

FIG. 18 shows successful leakage testing results over a 10-minute period, where the pressure changes by less than 0.1 PSI per minute after the first initial 30 seconds, which is the settling period. The control for cap 2 has a more drastic change than the others due to the cap not being fully sealed onto the assembly, but even with this mishap, the leak testing result was still successful.

TABLE 6

| | | |
|---|---|---|
| Number of insertions | Time (s) | Pressure (psi) |

Cap 1: 100 uL minivette pipette tip valve testing

| | | |
|---|---|---|
| 0 | 0 | 11.85 |
| | 0.25 | 11.82 |
| | 0.5 | 11.81 |
| | 0.75 | 11.8 |
| | 1 | 11.79 |
| | 1.25 | 11.78 |
| | 1.5 | 11.77 |
| | 1.75 | 11.77 |
| | 2 | 11.77 |
| | 3 | 11.75 |
| | 4 | 11.73 |
| | 5 | 11.72 |
| | 8 | 11.69 |
| | 10 | 11.66 |
| 1 | 0 | 13.43 |
| | 0.25 | 13.41 |
| | 0.5 | 13.39 |
| | 0.75 | 13.38 |
| | 1 | 13.38 |
| | 1.25 | 13.37 |
| | 1.5 | 13.36 |
| | 1.75 | 13.35 |
| | 2 | 13.35 |
| | 3 | 13.33 |
| | 4 | 13.31 |
| | 5 | 13.29 |
| | 8 | 13.26 |
| | 10 | 13.24 |
| 2 | 0 | 13.19 |
| | 0.25 | 13.17 |
| | 0.5 | 13.16 |
| | 0.75 | 13.15 |
| | 1 | 13.15 |
| | 1.25 | 13.14 |
| | 1.5 | 13.14 |
| | 1.75 | 13.13 |
| | 2 | 13.13 |
| | 3 | 13.12 |
| | 4 | 13.11 |
| | 5 | 13.1 |
| | 8 | 13.07 |
| | 10 | 13.05 |
| 3 | 0 | 12.39 |
| | 0.25 | 12.38 |
| | 0.5 | 12.37 |
| | 0.75 | 12.37 |
| | 1 | 12.36 |
| | 1.25 | 12.36 |
| | 1.5 | 12.35 |
| | 1.75 | 12.35 |
| | 2 | 12.35 |
| | 3 | 12.34 |
| | 4 | 12.33 |
| | 5 | 12.33 |
| | 8 | 12.31 |
| | 10 | 12.28 |
| 4 | 0 | 10.23 |
| | 0.25 | 10.21 |
| | 0.5 | 10.2 |
| | 0.75 | 10.19 |
| | 1 | 10.18 |
| | 1.25 | 10.18 |
| | 1.5 | 10.17 |
| | 1.75 | 10.17 |
| | 2 | 10.16 |
| | 3 | 10.15 |
| | 4 | 10.13 |
| | 5 | 10.12 |
| | 8 | 10.08 |
| | 10 | 10.06 |

Cap 2: 100 uL minivette pipette tip valve testing

| | | |
|---|---|---|
| 0 | 0 | 12.4 |
| Cap was not seated | 0.25 | 12.36 |
| on well initially | 0.5 | 12.34 |
| | 0.75 | 12.32 |
| | 1 | 12.31 |
| | 1.25 | 12.31 |
| | 1.5 | 12.3 |
| | 1.75 | 12.29 |
| | 2 | 12.28 |
| | 3 | 12.26 |
| | 4 | 12.23 |
| | 5 | 12.21 |
| | 8 | 12.17 |
| | 10 | 12.15 |
| 1 | 0 | 11.63 |
| | 0.25 | 11.62 |
| | 0.5 | 11.61 |
| | 0.75 | 11.6 |
| | 1 | 11.59 |
| | 1.25 | 11.59 |
| | 1.5 | 11.59 |
| | 1.75 | 11.58 |
| | 2 | 11.57 |
| | 3 | 11.56 |
| | 4 | 11.54 |
| | 5 | 11.53 |
| | 8 | 11.5 |
| | 10 | 11.49 |
| 2 | 0 | 13.19 |
| | 0.25 | 13.17 |
| | 0.5 | 13.16 |
| | 0.75 | 13.15 |
| | 1 | 13.15 |
| | 1.25 | 13.14 |
| | 1.5 | 13.14 |
| | 1.75 | 13.13 |
| | 2 | 13.13 |
| | 3 | 13.12 |
| | 4 | 13.11 |
| | 5 | 13.1 |
| | 8 | 13.07 |
| | 10 | 13.05 |
| 3 | 0 | 12.39 |
| A small, subtle amount | 0.25 | 12.38 |
| of leaking around the | 0.5 | 12.37 |
| tube fitting connection | 0.75 | 12.37 |
| was observed. | 1 | 12.36 |
| | 1.25 | 12.36 |
| | 1.5 | 12.35 |
| | 1.75 | 12.35 |
| | 2 | 12.35 |
| | 3 | 12.34 |
| | 4 | 12.33 |
| | 5 | 12.33 |
| | 8 | 12.31 |
| | 10 | 12.28 |
| 4 | 0 | 10.23 |
| Applied more epoxy, but | 0.25 | 10.21 |
| after some time a small, | 0.5 | 10.2 |
| subtle amount of leaking | 0.75 | 10.19 |
| around the tube fitting | 1 | 10.18 |
| connection was still observed. | 1.25 | 10.18 |
| | 1.5 | 10.17 |
| | 1.75 | 10.17 |
| | 2 | 10.16 |
| | 3 | 10.15 |
| | 4 | 10.13 |
| | 5 | 10.12 |
| | 8 | 10.08 |
| | 10 | 10.06 |

### Adding Shelf Feature to Insert and Input Port for Improvement of the Valve Seating

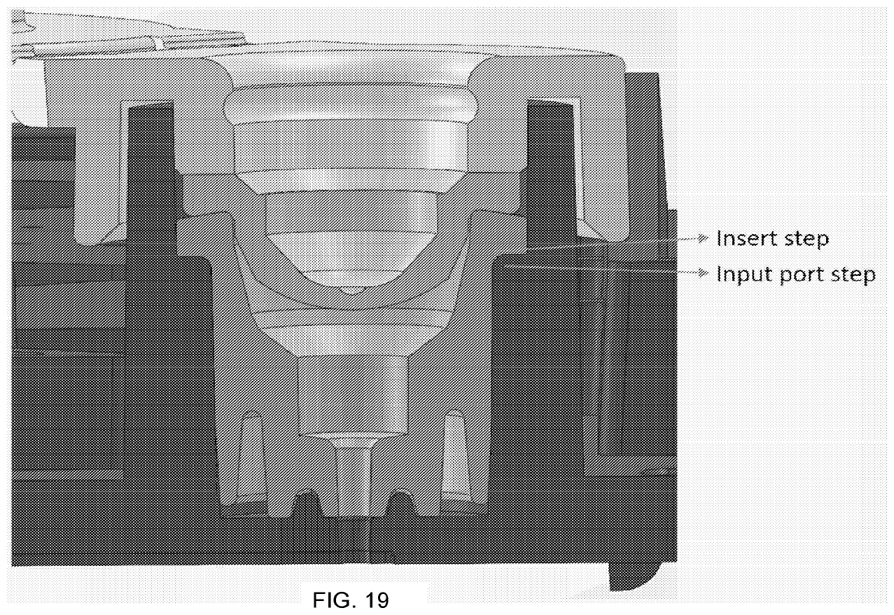

To further improve these leak testing results and ensure the consistency of valve seating, a step feature was added to the top of the input port. This feature would mate to the portion of the flexible insert that supports the valve's compression and seating in the assembly (FIG. 19). Without this step feature in the plastic of the input port, the arms of the flexible insert could shift downwards when a pipette tip was inserted, causing the valve to lose support and its ability to hold pressure. This additional change to the design ensures that the valve will always remain supported and compressed, while also not compromising the benefits supplied by the insert having compliant arms that prevent leakage and contribute to extra compliance and improved tactile feedback during pipette tip insertion.

### Rough Drawings of all Parts

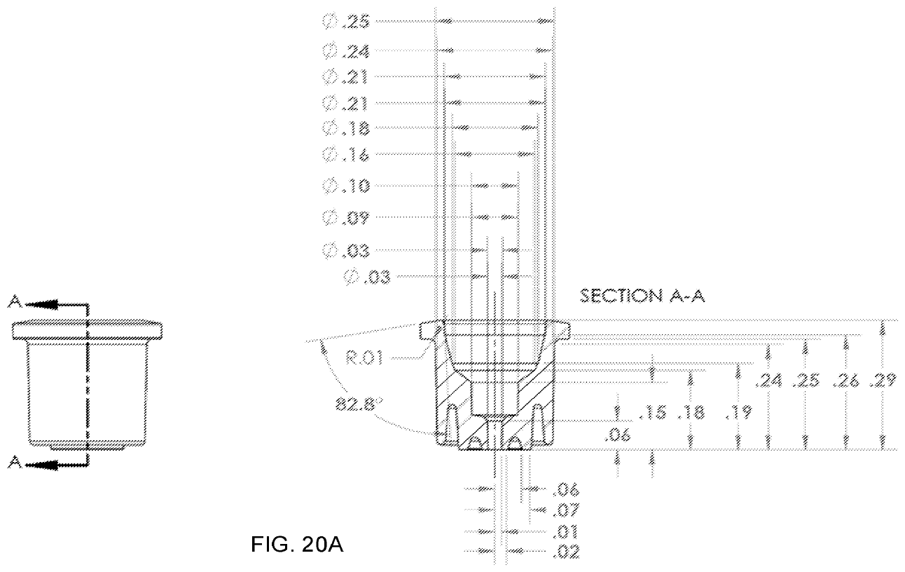

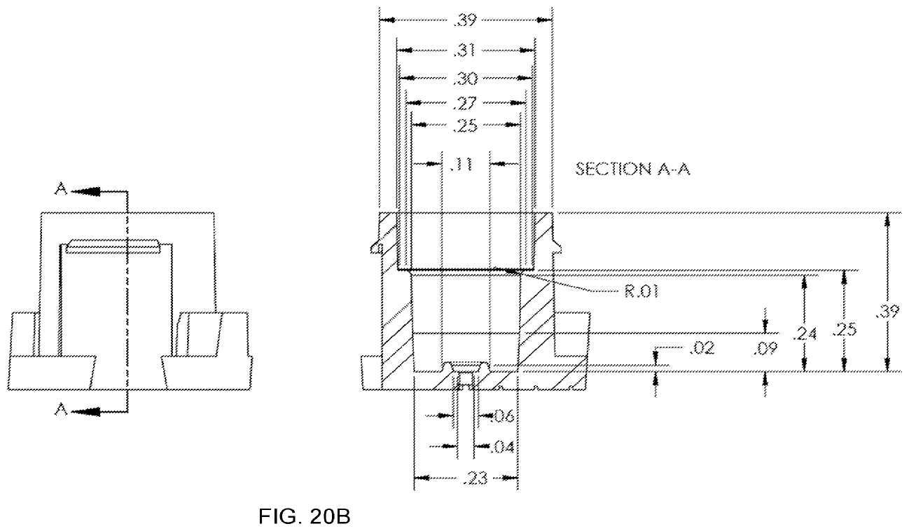

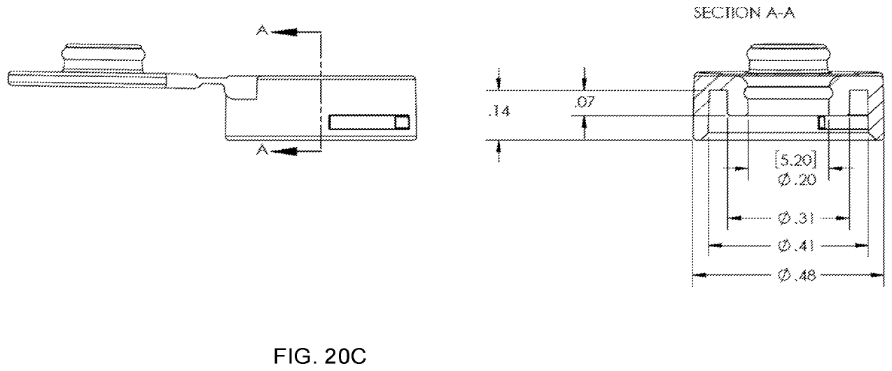

FIGS. 20A-C show the critical dimensions of each part within the assembly. The critical dimensions of the insert include the internal cavity dimensions associated with pipette tip engagement, the dead space directly below the valve, the dimensions associated with valve support as well as the step feature, the clearance on the lower ridge feature mated with the input port, and the taper of the arms as it press-fits into the cavity. The critical dimensions of the cap for the valve functionality include the height of the internal taper that compresses the valve. The critical dimensions of the input port include the taper along the input port's body, the step feature, and the height of the lower ridges.

Described herein is a thorough design and testing process to fulfill a critical need for improving user experience with inputting blood samples into an input port, such as an input port of a cartridge. The outcome of this process resulted in the design of a compliant insert that can be placed underneath a one-way valve for a user to blindly seal an instrument of different diameters into a central cavity at a variety of different insertion angles without needing to drastically increase the force of insertion. This insert and valve combination can be utilized to prevent backflow of liquid in high-pressure environments and closed systems, provides user with tactile feedback during insertion by providing both a guiding feature as well as a bottom lip that orients the insert itself around the mating cavity. For applications of microfluidics cartridge sample input, this design minimizes the dead space that may be associated with using different-sized pipette tips in one inlet port.

These requirements have been fulfilled by developing a compliant rubber insert with three cavity sections, a continuous internal taper to guide the pipette tip into the central cavity, an external arm feature for ease of insertion across different diameters and angles as well as a location for blood to pool up in case it leaks past the via hole onto the sides of the insert, a lower ridge for locating the central cavity of the insert with the via hole as well as another layer of defense for prevention of leakage through the sides. This design has been tested through prototyping, user testing on closed and open cartridge systems, and leakage testing using a fixture developed at CMT.

It will be appreciated that the devices and methods described above are set forth by way of example, and that the examples do not limit the scope of the invention. In addition, the order or presentation of method or assembly steps in the description above is not intended to require this order of performing the recited steps, unless a particular order is expressly required or is otherwise clear from the context. Thus, while this invention has been particularly shown and described with references to certain embodiments thereof, it will be understood in light of the present disclosure by those skilled in the art that various changes in form and details may be made therein without departing from the scope of the invention.

What is claimed is:

1. An input port assembly comprising an input port, a valve, and a cap, wherein the valve is oriented to be at a top portion of the input port, wherein the cap is located above the valve, and wherein the cap is tapered such that the bottom of the cap is narrower than the top of the cap.

2. The input port assembly according to claim 1, further comprising an O-ring located below the valve.

3. The input port assembly according to claim 1, further comprising two O-rings located below the valve.

4. An input port assembly comprising an input port, a valve, a cap, and a washer, wherein the valve is oriented to be at a top portion of the input port, wherein the cap is located above the valve, and wherein the washer is located in the interior space of the input port, and wherein said washer comprises an interior open cavity that is tapered such that the open cavity at the bottom of the washer is narrower than the open cavity at the top of the washer.

5. The input port assembly according to claim 4, wherein the washer is a foam washer.

6. An input port assembly comprising an input port, a valve, a cap, and an insert, wherein the valve is oriented to be at a top portion of the input port, wherein the cap is oriented to be on top of the valve, and wherein the insert is located in the interior space of the input port, and wherein the insert comprises an open cavity and allows for the insertion of an instrument into the open cavity, and wherein, at the bottom of the open cavity is an opening leading to the input port.

7. The input port assembly according to claim 6, wherein the insert is a rubber insert.

8. The input port assembly according to claim 7, wherein the rubber insert is press-fit into the interior space of the input port.

9. The input port assembly according to any one of claims 6-8, wherein the rubber insert comprises:
(i) a first region located at the top of the insert, wherein the first region comprises a one-way slit valve that is configured to open when a pipette tip is inserted;
(ii) a second region located underneath the first region, wherein the second region comprises an interior open cavity; and
(iii) a third region located underneath the second region, wherein the third region comprises an interior open cavity that is narrower than the second region's interior open cavity, across the height of the third region.

10. The input port assembly according to any one of claims 6-9, wherein the instrument is a pipette tip.

11. An input port assembly comprising an input port, a one-way valve, a cap, and an insert, wherein the valve is oriented to be at a top portion of the input port, wherein the cap is oriented to be on top of the valve, and wherein the insert is located in the interior space of the input port, and wherein the insert comprises a central cavity, and wherein:
a. the central cavity comprises a first section, a second section, and a third section;
b. the first section is associated with a space for the one-way valve to open when an instrument is inserted;
c. the second section is located underneath the valve; and
d. the third section is underneath the second section, and wherein in the third section, the diameter of the central cavity, at all points along the height of the third section, is smaller than the diameter of the central cavity in the second section, at all points along the height of the second section.

12. The input port assembly according to claim 11, further comprising one or more arms that are located outside of the central cavity.

13. The input port assembly according to any one of claims 11-12, wherein the insert is a compliant insert.

14. The input port assembly according to any one of claims 11-12, wherein the insert is a rubber insert.

15. An insert comprising a central cavity, wherein the interior space of the central cavity tapers towards the bottom of the insert, and wherein the insert further comprises one or more arms.

16. The insert according to claim 15, wherein the insert is made of a compliant material.

17. The insert according to claim 15, wherein the insert is a rubber insert.
